(19) United States
(12) Patent Application Publication
(10) Pub. No.: US 2007/0074326 A1
(43) Pub. Date: Apr. 5, 2007

(54) Headgear With Cooling Device

(75) Inventor: Cindy Komechak, Ozona, TX (US)

Correspondence Address: SACHNOFF & WEAVER, LTD., 10 SOUTH WACKER DRIVE, CHICAGO, IL, 60606-7507, US

(73) Assignee: Koolin Klothz, Etc., Ozona, TX (US)

(21) Appl. No.: 11/469,451

(22) Filed: Aug. 31, 2006

Related U.S. Application Data
(60) Provisional application No. 60/713,412, filed on Sep. 1, 2005.

(51) Int. Cl. A61F 9/00

(52) U.S. Cl. 2/12

(57) ABSTRACT

The present invention is directed to headwear including a cooling device formed using a woven or non-woven material. More particularly, the invention relates to headwear including a cooling device formed of an absorptive material such as polyvinyl alcohol (PVA) having high absorptive capacity. The headwear and the cooling device are designed to fit securely on the wearer's head or with a hat. When moistened, the cooling device provides sustained cooling via an evaporative cooling process.

### RELATED DOCUMENTS

Incorporated herein by reference, this application is a continuation of and claims priority to U.S. Provisional Application No. 60/713412 filed on Sep. 1, 2005 and entitled, “Headgear with Cooling Device”.

### BACKGROUND

The present invention generally relates to headwear including a cooling device made of cloth or cloth-like material. More particularly, the invention relates to headwear including a cooling device having a surface, which when moistened, facilitates cooling of a surface via evaporative cooling.

There are many types of headwear or head coverings used or designed primarily for sporting activities. Aside from protective helmets, one of the most common types of head coverings is a cap. Among these, the baseball cap is popular. Another type of headwear used in some sporting activities, such as tennis and soccer, is a sweatband. Still another type of headwear is a do-rag. The do-rag is typically a piece of fabric, generally in the shape of a square, which is adapted to fit the wearer's head by simply knotting it at the corners so as to fit the head of the wearer and to cover the hair and, in some cases, the upper portion of the wearer's head, including the left and right sides just above the ears.

While headwear has been designed and used for a wide variety of practical purposes, including for instance, to protect the head from physical injury, rain or snow, there is a need for highly portable, washable and durable headwear that can very easily provide a prolonged cooling effect to the wearer's head.

### SUMMARY OF THE INVENTION

The present invention is directed to headwear including a cooling device formed using a woven or nonwoven material. More particularly, the invention relates to headwear including a cooling device formed of an absorptive material. The headwear and the cooling device are designed to fit securely on the wearer's head. When moistened, the cooling device provides sustained cooling via an evaporative cooling process.

### BRIEF DESCRIPTION OF THE DRAWINGS

This patent is illustrated by way of examples and not limitations in the accompanying figures, in which like references indicate similar elements, and in which:

FIG. 1A illustrates an embodiment of headwear in the form of a do-rag which includes a cooling device in accordance with the teachings of this invention;

FIG. 1B illustrates an alternate embodiment of headwear in the form of another do-rag which includes cooling device in accordance with the teachings of this invention;

FIG. 2A illustrates an interior view of the headwear and the cooling device shown in FIG. 1A;

FIG. 2B illustrates an interior view of the headwear and the cooling device shown in FIG. 1B;

FIG. 2C illustrates an interior view of an embodiment of headwear, in the form of a canopy and cooling device in accordance with the teachings of this invention;

FIG. 2D illustrates a side exterior view of the headwear including the cooling device shown in FIG. 2C;

FIG. 3 illustrates the cooling device shown in FIGS. 2A and 2B;

FIG. 4 illustrates a side exterior view of the cooling device shown in FIG. 3;

FIG. 5 illustrates two cooling device embodiments inserted into a hat and a cap, respectively;

FIG. 6A illustrates a cooling device embodiment that affixes to the interior of a hat.

FIG. 6B illustrates a convertible cooling device pouch embodiment that affixes to the interior of a hat.

FIG. 7A illustrates headwear embodiment in the form of a bandanna, which includes the cooling device in accordance with the teachings of this invention;

FIG. 7B illustrates an interior view of the headwear and the cooling device shown in FIG. 7A; and,

FIG. 8 illustrates a headwear embodiment in the form of a band, which includes the cooling device in accordance with the teachings of this invention.

### DETAILED DESCRIPTION

Illustrative embodiments of headwear including a cooling device formed in accordance with the teaching of the invention are described herein with reference to the accompanying drawing figures. The headwear and the cooling device can be adapted to take any shape to cool any surface or any exposed body part or a portion thereof. One of ordinary skill in the art will recognize that the present invention may be practiced in a variety of spatial orientations without departing from the spirit and scope of the invention.

The cooling device may be formed of any highly porous cloth or cloth-like material that reacts with water to create a cooling effect. For example, it may be formed of polyvinyl alcohol (PVA). The cooling device may be, depending on the material used, hydrophilic, hydroscopic, chemically resistant, resilient, machine washable, and/or colorable. The cooling device may also be formed into any desired shape, such as do-rags, headwear inserts, hat inserts, bandanna inserts, bands (e.g., headbands and wristbands), etc.

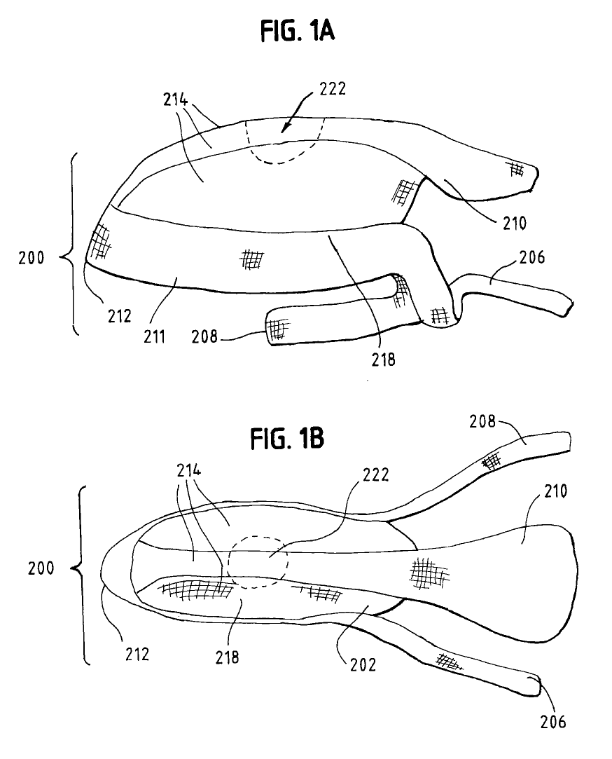

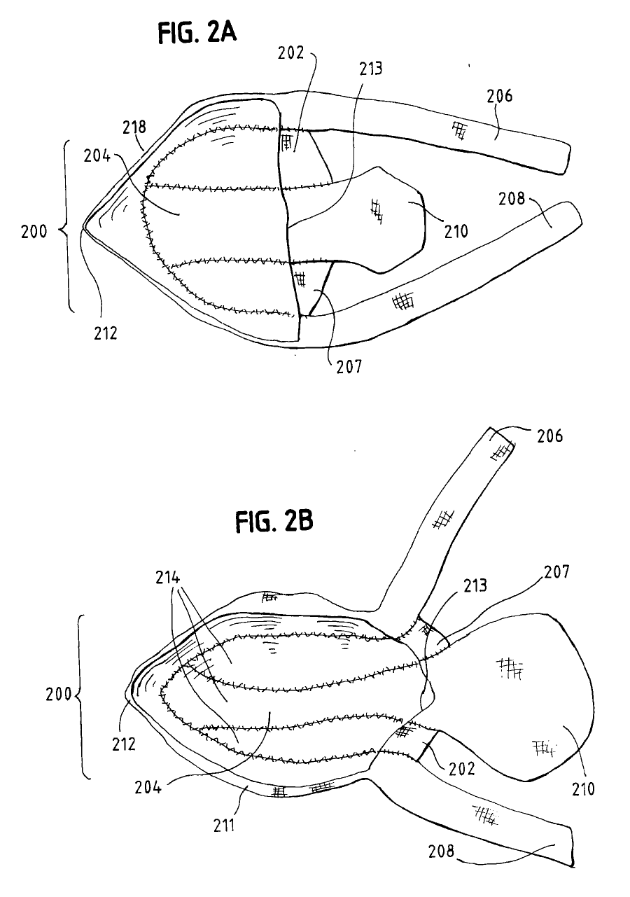

In one embodiment of the invention, as best seen in FIGS. 1A and 2A, headwear embodiments may be configured as varying do-rags 200. The do-rag 200 includes a body portion 202, a headwear insert 204 and two ties 206, 208. The body portion 202 also includes a tail 210, as best seen in FIG. 2A. In the one embodiment, the body portion 202 is made of any suitable woven material, for example a natural fiber, a synthetic fiber, or combinations thereof.

In one embodiment, as best seen in FIG. 2A, the body portion 202 includes a surface 218 configured in a triangular-like shape. In the embodiment shown in FIG. 1A, the surface 218 of the body portion 202 includes three (3) panels 214 having an arcuate shape and a forward panel 211. The three (3) panels 214 of arcuate shape are sewn together along their length, and the forward panel 211 is sewn onto the three (3) panels 214 so as to create a band that surrounds the front and side portions of the assembled three (3) panels 214.

In one embodiment, as shown in FIGS. 1A-1B, the three (3) panels 214 of the body portion 202 when sewn together create a cup-like configuration that defines a crown covering portion or a crown portion 222 of the do-rag 200. The crown portion 222 sits on the wearer's head like a skull cap, covering a portion of the top, rear, and left and right sides of the wearer's head.

In one embodiment best seen in FIG. 1A, the forward panel 211 when sewn onto the three (3) panels 214 of arcuate shape forms a forward edge 212 of the body portion 202 designed to cover at least a portion of the forehead of the wearer. The forward edge 212 may be extended to a length that stops just above the wearer's ears, particularly if the wearer is a human user.

In one embodiment, as best seen in FIG. 2A, the body portion 202 includes a rear edge 213 that is adapted for positioning against the nape of the wearer's neck. In the one embodiment, as shown in FIG. 2A, an exposed interior 207 of the body portion 202 is extended to allow the headwear to fall lower on the wearer's neck. The body portion 202 also supports the ties 206, 208 that extend out from the body portion 202 at the forward edge 212.

In one embodiment, as shown in FIGS. 1A and 2A, the ties 206, 208 are configured as extensions of the band forming the forward edge 212. The ties 206, 208 are elongated to allow them to be tied together, thus, permitting adjustment of the body portion 202 about the wearer's head. The ties 206, 208 are preferably configured so that the ties 206, 208 may be secured to each other behind the head of the wearer at the nape of the neck.

Whether the body portion 202 is secured by the ties 206, 208, as shown in FIGS. 1A and 2A, or a fastening element, the body portion 202 is configured to be adjustable so that the body portion 202 may be conveniently and snugly fitted and fastened to any head size.

In one embodiment, as best seen in FIG. 2A, the body portion 202 includes the tail 210. The tail 210 extends beyond the body portion 202 so as to cover at least a portion of the wearer's neck. In the embodiment shown in FIGS. 1A and 2A, the tail 210 and at least one of the three (3) panels 214 are formed from a single sheet of material.

In one embodiment, as shown in FIG. 2A, the body portion 202 supports the headwear insert 204. As shown in FIG. 2A, the headwear insert 204 abuts the forward edge 212 of the body portion 202, and is configured having a shape that conforms to the shape of the crown of the wearer's head. For example, the headwear insert 204 shown in FIG. 2A is configured to fit over the crown of the head of a human wearer. In one embodiment, the headwear insert 204 is attached to the body portion 202 by stitching and the like.

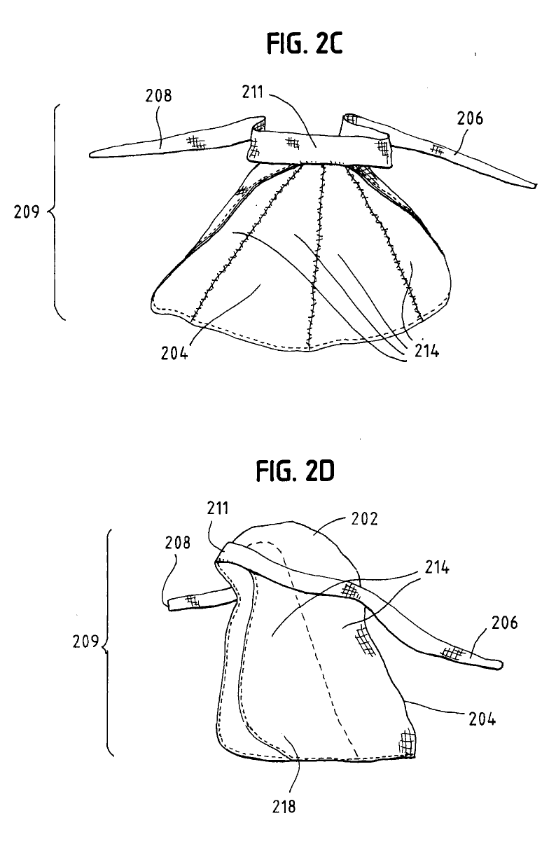

In the one embodiment shown in FIG. 2A, the headwear insert 204 is formed using polyvinyl alcohol (PVA), a hydrophilic material. In the embodiment as best seen in FIG. 3, the headwear insert 204 is cut and sewn to allow the device to fit closely over the surface to be cooled.

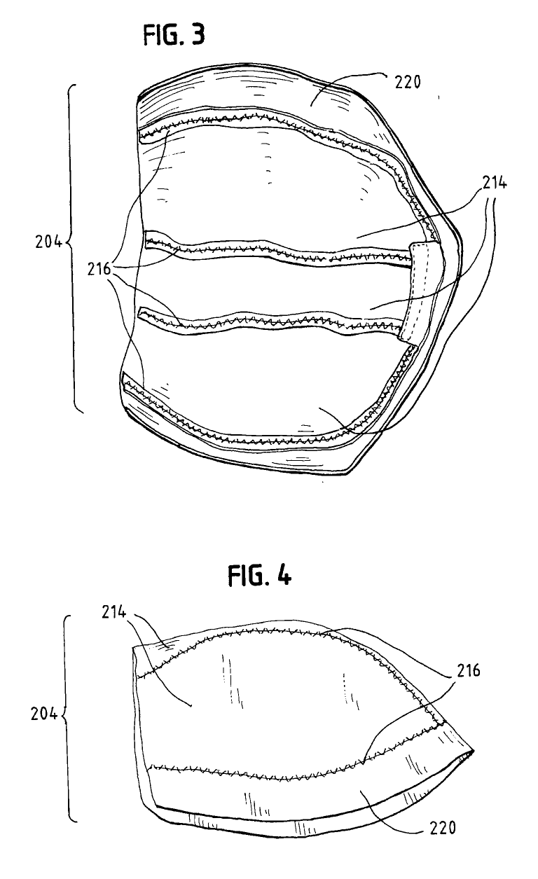

In the embodiment shown in FIGS. 3 and 4, one method of forming the headwear insert 204 is to sew the three (3) panels 214 of PVA material, for example, to one another as illustrated by stitch lines 216. The assembled three (3) panels 214 of the headwear insert 204 are then sewn to a band portion 220 of the headwear insert 204. To achieve the desired shape the three (3) panels 214 and the band portion 220 are cut in a configuration that allows the completed headwear insert 204 to match the contour of the surface to be cooled. For example, the embodiment of the headwear insert 204 shown in FIG. 3 is designed to fit over the crown of the head of a human wearer.

Several additional embodiments of the foregoing headwear with cooling device have been contemplated. For example, while the above described body portion 202 of the headwear with cooling device is configured in the triangular-like shape, in an alternate embodiment, the body portion 202 may be configured in a variety of shapes and sizes, including but not limited to rectangular, triangular and pentagonal. However other suitable shapes will be readily apparent to those of ordinary skill in the art from the present disclosure, and the present invention is not limited in any way to the shape of the body portion 202 of the illustrated embodiments. Additionally, in an alternate embodiment, the body portion 202 may be made of any suitable woven or non-woven material, for example a natural fiber, a synthetic fiber, leather, plastic, or combinations thereof.

While the exposed interior 207 of the body portion 202 shown in FIG. 2A is extended, one of ordinary skill in the art will appreciate that the exposed interior 207 of the body portion 202 may vary in size. For example, in an alternate embodiment of the headwear also configured as a do-rag, shown in FIG. 2B, the exposed interior 207 of the body portion 202 is minimal. Additionally in this alternate embodiment, the sides of the forward panel 211 are shortened so as not to cover the wearer's ears. One of ordinary skill in the art will appreciate that the forward panel 211, and optionally the three (3) panels 214, may vary in size suitable for its intended purpose.

While the surface 218 of the body portion 202 shown in FIG. 1A is formed by sewing the forward panel 211 onto the three (3) panels 214 of arcuate shape, one of ordinary skill in the art will appreciate that there are various methods for forming the surface 218, and the invention is not to be limited by the method of forming the illustrated embodiment. For example, in an alternate embodiment shown in FIGS. 2C and 2D, the headwear may be in the form of a canopy 209. In this alternate embodiment, the forward panel 211 may abut against the wearer's forehead, and the PVA material, for example, may be located inside at least a portion of the forward panel 211. One of ordinary skill in the art will appreciate that the PVA material, for example, may be located inside at least one of the panels 214 of the body portion 202, and optionally the forward panel 211, or attached to the body portion 202 and combinations thereof. The body portion 202 may support the headwear insert 204. In this embodiment, the headwear insert 204 may be formed by sewing four (4) panels 214 together as shown in FIG. 2C. Additionally, in this alternate embodiment, the three (3) panels 214 of the body portion 202 (FIG. 2D) and the four (4) panels 214 of the headwear insert 204 (FIG. 2C) may extend to the wearer's shoulders, serving to protect not only the head, but also the ears and neck from cancer-causing ultraviolet sun radiation. This is accomplished not just by covering the head, ears and neck with the body portion 202, but also by covering the head, ears and neck with the headwear insert 204, all while providing a cooling effect. Thus, the canopy 209 may serve as enhanced ultraviolet blocking protective headwear for medical patients, healthy children, adults, or elderly persons seeking shelter from damaging sun rays. One of ordinary skill in the art will appreciate that the surface 218 of the body portion 202 and the headwear insert 204 may be formed from one or more panels cut to the desired configuration and attached in the desired configuration suitable for its intended purpose.

While the ties 206, 208 shown in FIGS. 1A and 2A are configured to be secured to each other behind the head of the wearer at the nape of the neck, in an alternate embodiment shown in FIGS. 2C and 2D, the ties 206, 208 may be secured in the back of the wearer's head. One of ordinary skill in the art will appreciate that the ties 206, 208 may vary in length (shown in FIGS. 2B and 2C) and may be secured in a variety of locations around the head and the neck of the wearer. Additionally, while the ties 206, 208 as shown in FIGS. 1A and 2A are configured as extensions of the band forming the forward edge 212, in an alternate embodiment, the ties 206, 208 may be sewn onto the body portion 202. One of ordinary skill in the art will appreciate that other methods may be used to attach the ties 206, 208 to the body portion 202. One of ordinary skill in the art will also appreciate that the ties 206, 208 may support a fastening element, such but not limited to, one or more hook and loop fastener(s), snap(s), latch fastener(s), button(s), a zipper, or hook and loop fastener, or any known fastener and combinations thereof, that permit adjustment of the body portion 202 about the wearer's head may be used.

While the tail 210 and at least one of the three (3) panels 214 of FIGS. 1A and 2A are formed from a single sheet of material, in an alternate embodiment, the tail 210 may be sewn onto the rear edge 213, and PVA material may be located inside the tail 210. One of ordinary skill in the art will appreciate that other methods may be used to attach the tail 210 to the body portion 202, and that the tail 210 may be attached without PVA material. Furthermore, the tail 210 and the body portion 202 may be formed from a single sheet of material. One of ordinary skill in the art will also appreciate that the tail 210 may be configured in a variety of shapes and sizes suitable for its intended purpose. In an alternate embodiment shown in FIGS. 2C and 2D, the tail 210 may not be present.

While the headwear insert 204 of FIG. 2A is attached to the body portion 202 by stitching and the like, in an alternate embodiment, the headwear insert 204 may be formed integrally with the body portion 202. If the headwear insert 204 and the body portion 202 are integrally formed, the body portion 202 and the headwear insert 204 will be formed using a material having hydrophilic and/or hydroscopic properties. One of ordinary skill in the art will appreciate that the PVA material, for example, may also be located inside at least a portion of the body portion 202 or the tail 204 or combinations thereof.

While the headwear insert 204 of FIGS. 3 and 4 has been described having the three (3) panels 214 of the PVA material, for example, sewn to the band portion 220 of the PVA material, for example, one of ordinary skill in the art will appreciate that other methods may be used for forming the headwear insert 204.

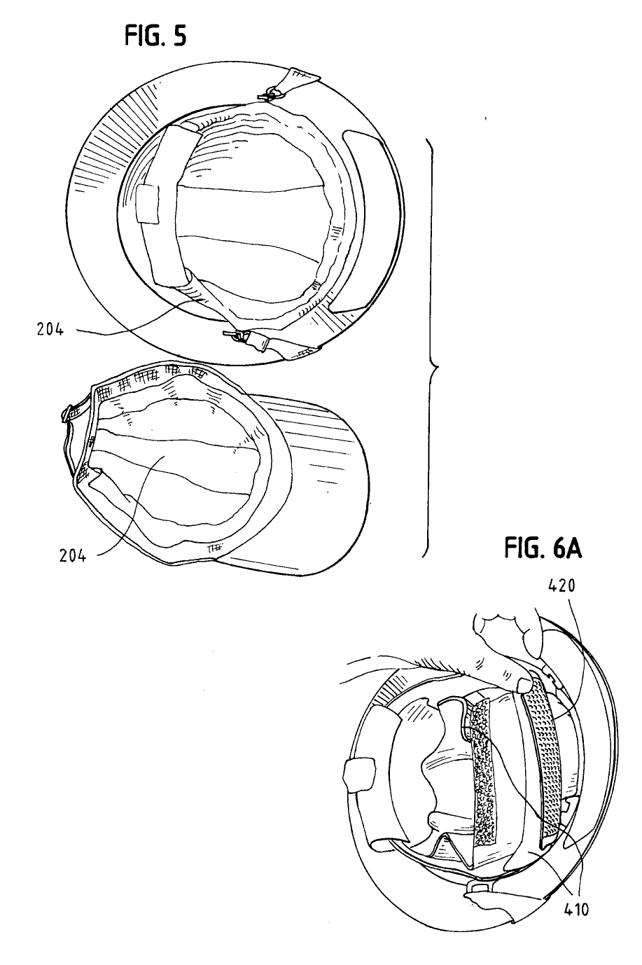

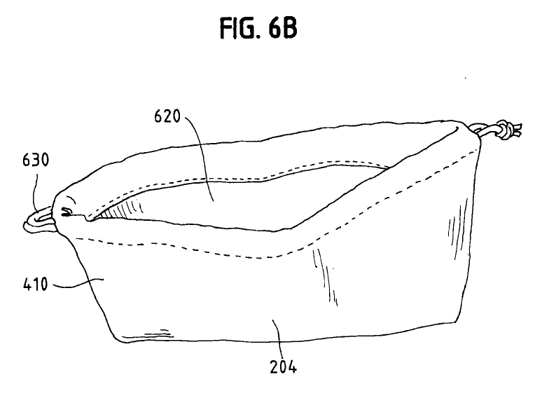

In an alternate embodiment best seen in FIG. 5, the headwear insert 204 may be configured as a baseball cap insert 400 or a hat insert 420. FIG. 6A further shows the hat insert 420 placed inside a hat. Both the hat insert 420 and the baseball cap insert 400 may be attached to the surfaces within the hat/cap via hook and loop fastener strips 410. In still another embodiment shown in FIG. 6B, the headwear insert 204 may be convertible into a pouch, having a resealable opening 620 and hook and loop fasteners 410 (not visible). The resealable opening 620 may be resealable by any known way, including a drawstring 630. The headwear insert 204 embodiment of FIG. 6B may be used not only to cool the wearer's head, but also to store items within the hat which may be kept cool. Alternatively, the convertible insert may be used to cool the head until such time as it may be needed as a pouch for carrying items outside the hat in cooled, moist environment.

One of ordinary skill in the art will appreciate that the inserts 204 may be attached and reattached by a variety of fasteners in place of or in conjunction with hook and loop fastener strips 410, including snap(s), latch fastener(s), button(s), a zipper, or ties or equivalents or combinations thereof formed according to the teachings of this invention. Similarly, one of skill in the art will appreciate that the resealable opening 620 may be resealed using the foregoing fasteners or equivalents alone or in combination.

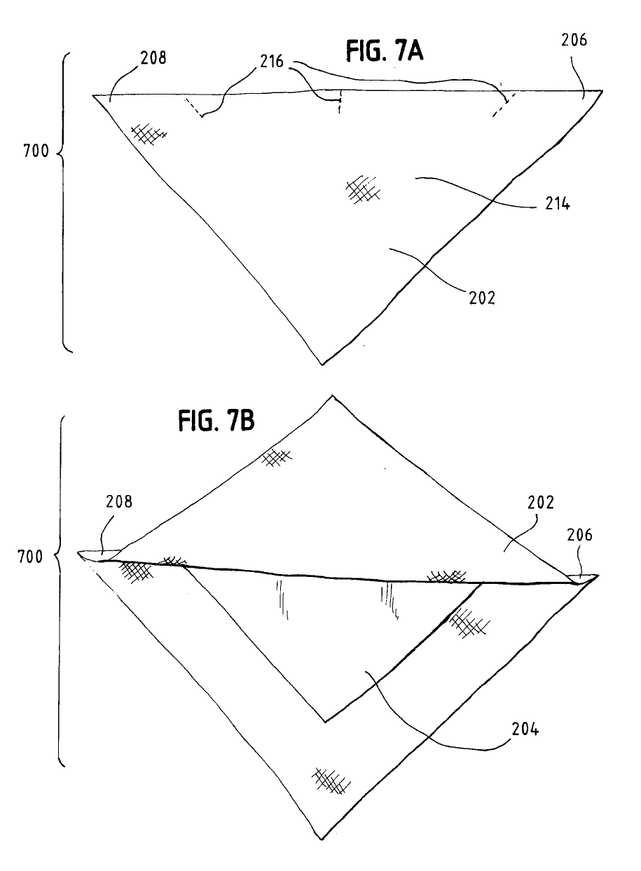

In an alternate embodiment shown in FIGS. 7A and 7B, the headwear may be in the form of a bandanna 700. The bandanna 700 may include the body portion 202, the headwear insert 204 of the PVA material, for example, and the ties 206, 208, shown in FIGS. 7A and 7B. The bandanna 700 with the cooling device may act as a convertible cooling mask for air filtration and heat stress relief. Particularly, the bandanna 700 with the cooling device, when placed over the wearer's nose and mouth, substantially prevents dust and hot air from being inhaled. In this alternate embodiment, the headwear insert 204 may be formed from the single panel 214 of the PVA material, for example, in a triangular-like shape lesser than or equal to the dimensions of the bandanna 700, as seen in FIG. 7B. Further in this embodiment, the bandanna 700 may form a triangular shape, as best seen in FIG. 7A. In this alternate embodiment, one method of attaching the headwear insert 204 may be to sew the single panel 214 of the PVA material, for example, to the bandanna as illustrated by the stitch lines 216 in FIG. 7A. Further in this embodiment, the body portion 202 includes the ties 206, 208, which can be secured around the wearer's head. One of ordinary skill in the art will also appreciate that the ties 206, 208 may support a fastening element, such but not limited to, one or more hook and loop fastener(s), snap(s), latch fastener(s), button(s), a zipper, or hook and loop fastener, or any known fastener and combinations thereof, that permit adjustment of the bandanna 700 about the wearer's head may be used. In an alternate embodiment, the bandanna 700 with the headwear insert 204 may be converted to a band by folding the bandanna 700 to a desired configuration to cool the surface in contact with the band, i.e., the wearer's forehead, wrist, ankle, and body.

One of skill in the art will further appreciate that an alternate embodiment of the bandanna 700 may be formed using alternative fashion-quality fabrics, such as, for example, silk or silk substitutes to create designer scarves. Such scarves could be readily converted merely by wetting to immediately treat heat stress. For example, a woman experiencing hot flashes could wet her fashion scarf and apply it to the forehead, face, neck, arms or any other afflicted area to provide immediate heat stress relief.

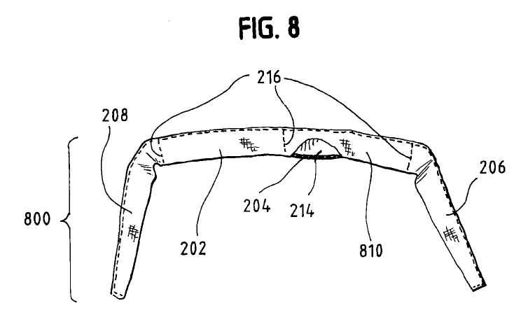

In an alternate embodiment shown in FIG. 8, the headwear may be in the form of a band 800 made of any suitable woven material. One of ordinary skill in the art will appreciate that the band 800 may be made of any suitable woven or non-woven material, for example a natural fiber, a synthetic fiber, leather, plastic, or combinations thereof. The band 800 may include the headwear insert 204, the body portion 202 and the ties 206, 208. To create the band 800, the band material of the body portion 202 may be formed into a tube like structure having an outer surface 810 and the ties 206, 208. The outer surface 810 of the band 800 may directly contact the surface to be cooled. The headwear insert 204 may be formed from the single panel 214 of the PVA material, for example, in a rectangular-like shape, for example, of equal of lesser dimensions than the band 800. In this alternate embodiment, one method of attaching the headwear insert 204, which forms an inner layer of the band 800, may be to sew the single panel 214 of the PVA material, for example, to the band 800 as illustrated by the stitch lines 216 in FIG. 8. In the alternate embodiment, the body portion 202 of the band 800 extends beyond the headwear insert 204 to form the ties 206, 208 to secure the band in place. One of ordinary skill in the art will also appreciate that the ties 206, 208 may support a fastening element, such but not limited to, one or more hook and loop fastener(s), snap(s), latch fastener(s), button(s), a zipper, or hook and loop fastener, or any known fastener and combinations thereof, that permit adjustment of the band 800 about the wearer's head, wrist, ankle, and body, for example, may be used. In an additional alternate embodiment, the ties 206, 208 may be sewn onto the body portion 202 of the band 800. One of ordinary skill in the art will appreciate that other methods may be used to attach the ties 206, 208 to the body portion 202. In all the foregoing figures and examples, the surfaces to be cooled by the cooling device are not limited to the human exterior.

One of skill in the art will appreciate that an embodiment of the band 800 may be easily formed from the bandanna 700, yet provide one-and-a-half to three times the absorption of the band 800 due to the relatively larger surface area of the bandanna 700 insert 204. By either folding or rolling the exceptionally lightweight bandanna 700, the bandanna 700 may be converted, for example, by fire safety personnel into a super-wicking embodiment of the band 800. In extreme fire-fighting conditions, where temperatures can be extraordinarily high, the rolled bandanna 700, with its multiple layers of PVA material, may serve as a powerful wicking band, used to quickly absorb large amounts of perspiration from the forehead, thereby allowing fire-fighters to see more clearly and work more safely, simply by keeping running perspiration out of the eyes.

While the description above refers to particular embodiments of the present invention, it will be understood that many modifications may be made without departing from the spirit thereof. The accompanying claims are intended to cover such modifications as would fall within the true scope and spirit of the present invention. The presently disclosed embodiments are therefore to be considered in all respects illustrative and not restrictive, the scope of the invention being indicated by the appended claims, rather than the foregoing description, and all changes which come within the meaning and range of equivalency of the claims are therefore intended to be embraced therein.

What is claimed is:

1. A cooling device comprising:
a first panel made of a cloth material;
a second panel attached to the first panel and made of an absorptive material; and
wherein the first panel and the second panel are shaped in a manner such that the first panel may be folded to cover the second panel on both sides.

2. The cooling device of claim 1, wherein the first panel is made of cotton.

3. The cooling device of claim 2, wherein the second panel is made of polyvinyl alcohol (PVA).

4. The cooling device of claim 3, wherein:
shape of the first panel approximates a rectangle;
shape of the second panel approximates a triangle; and
the base of the second panel is attached to the diagonal of the first panel.

5. The cooling device of claim 2, wherein the second panel is made of a material having at least one of hydroscopic property and hydrophilic property.

6. A cooling device comprising:
a plurality of panels attached together to approximate shape of crown of a human head;
at least one of the plurality of panels being made of polyvinyl alcohol;
at least one of the plurality of panels having a bottom fastening element attached to it; and
at least one of the plurality of panels having an attachment loop attached to it, the attachment loop having a top fastening element attached to it.

7. The cooling device of claim 6, wherein the bottom fastening element is one flap of a hook and loop fastener and the top fastening element is the mating flap of the hook and loop fastener.

8. The cooling device of claim 6, wherein the attachment loop is designed to attach the cooling device to an adjustment strip of a hat.

9. The cooling device of claim 6, wherein the bottom fastening element is a male end of a snap on button and the top fastening element is the female end of the snap on button.

10. An assembly of a hat and a cooling device, the assembly comprising:
the hat having an inner strip attached near a bottom edge of the hat;
the cooling device made of a plurality of panels joined together in a shape corresponding to inner surface of the hard hat; and
the cooling device having an attachment mechanism that is designed to attach the cooling device to the inner strip of the hat.

11. The assembly of claim 10, wherein the cooling device is made of polyvinyl alcohol.

12. The assembly of claim 10, wherein the attachment mechanism is made using two flaps of Velcro™ strip.

13. The assembly of claim 10, wherein:
the cooling device further includes a first flap of a Velcro™ strip attached to the outer surface of the cooling device;
the hat further includes a second flap of the Velcro™ strip attached to the inner surface of the hat; and
the first flap and the second flap are positioned in a manner to allow the cooling device to attach to the inner surface of the hat.

14. The assembly of claim 10, further comprising:
a pouch made of polyvinyl alcohol, the pouch having an outer attachment mechanism on its outer surface; and
the hat having an inner attachment mechanism on its inner surface that attaches to the outer attachment mechanism.

15. A cooling device comprising:
a cooling insert comprising a plurality of panels attached together to approximate shape of crown of a human head;
at least one of the plurality of panels being made of polyvinyl alcohol;
outer surface of the cooling insert attached to a cloth material; and
the cloth material having a two side flaps on the opposite lower ends of the inner insert and extending beyond the surface area of the inner insert.

16. The cooling device of claim 15, wherein the cloth material further comprising a bottom flap between the two side flaps and extending beyond the bottom of the inner insert.

17. The cooling device of claim 16, wherein the plurality of panels includes three arcuate panels sown to each other and a fourth panel attaching the front part of the crown shape.

18. The cooling device of claim 17, wherein the bottom flap and the fourth panel are on the opposite sides of each other.

19. A headband comprising:
an inner strip of water absorptive material made of polyvinyl alcohol (PVA); and
the inner strip covered by an outer tube like housing.

20. The headband of claim 19, wherein the housing is made of at least one of: (1) natural fiber; (2) synthetic fiber; (3) leather and (4) plastic.

21. The headband of claim 20, wherein the housing having a first end and a second end and each of the first and second ends of the housing extends beyond the inner strip to form a tie such that the first end and the second end may be tied to each other.

22. The headband of claim 21, further comprising:
a first fastening element attached to the first end of housing and a second fastening element attached to the second end of the housing, the first fastening element and the second fastening elements adapted to be fastened to each other.
